(19) United States
(12) Patent Application Publication
(10) Pub. No.: US 2009/0181084 A1
(43) Pub. Date: Jul. 16, 2009

(54) Oral fast-disintegrating tablet including particles of slowly-releaseable ascorbic acid

(76) Inventor: Ta-Ping Liao, Chia Yi City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/007,714

(22) Filed: Jan. 15, 2008

(51) Int. Cl. A61K 9/24; A61K 31/375

(52) U.S. Cl. 424/465; 424/472; 514/474

(57) ABSTRACT

An oral fast-disintegrating tablet includes many tiny particles of slowly-releasable ascorbic acid, and a fast-disintegrating ingredient mixed with the many tiny particles that are dispersedly mixed with the fast-disintegrating ingredient. Each tiny particle is composed of a nucleus, an ascorbic layer and a release-control layer. The nucleus is coated with the ascorbic-acid layer and then with the releasing control layer to become a slowly-releasable particle. The tiny particles are mixed with the fast-disintegrating ingredient, which is composed of a mixture of ascorbic acid, water excipient, microcrystalline cellulose, a diluent agent, and disintegrant. So the tablet can be disintegrated swiftly in a mouth, and the tiny particles can enter the stomach and intestines with saliva to be slowly released, sustainingly absorbed by the organs.

### BACKGROUND OF THE INVENTION

1. Field of the Invention

This invention relates to an oral disintegrating tablet including particles of slowly-releasable ascorbic acid, particularly to one composed of several hundreds of tiny particles dispersedly mixed with fast-disintegrating ingredient, and possible to dissolve in a mouth to release the tiny particles that can be moved with saliva into the stomach and intestines and let the ascorbic acid (vitamin C) contained in the tiny particles to slowly release from a nucleus, sustaining a long period of time to be absorbed by the organs of a human body.

2. Description of the Prior Art

Vitamin C also called L-ascorbic acid is an indispensable nutrient for highly intellectual animals and a few creatures, and its medicinal base is ascorbic acid. Vitamin (C is a highly effective anti-oxidant used for reducing oxidative stress of ascorbate peroxidase so as to protect a human body from the threat coming from oxidizers.

Aside from the function of anti-oxidization in a human body, vitamin C is related with a lot of biochemical reactions in a human body. For an example, vitamin C can advance formation of collagen, which exists in the connective issues, the systems of blood vessels, bones, tooth cells, having a more mechanical strength than that of iron and steel, being a basic supporting substance of animal bodies. Hence, it can permit cells tightly connected together, the skin extend tensely, and bones and teeth hard and solid. If a human body is wounded or receives an operation, collagen assists cells to restore, and boosts recovery of a trauma.

On the contrary, in case of lack of collagen, the reactions just described above may not be carried out smoothly, and many related organs may suffer from diseases. One of the well known cases is extremely lack of vitamin C, which may lead to scurvy, and reduced volume of vitamin C can cause instability of collagen in some systems, so collagen cannot perform its usual function. Scurvy has symptoms of red spots on the skin, sponge-like tooth roots, and oozing of blood from mucous membranes, and especially red spots may appear mostly on the thighs. Patients suffering from scurvy may have a pale face and be depressed, and some may be quite impossible to walk around solely. Open wounds of ulcer may be involved in case of heavy scurvy, and teeth may fall off. Therefore, human bodies have to timely take vitamin C to prevent such diseases described above.

However, human bodies cannot store vitamin C, so it must be taken regularly and continually, or it can be used up soon, and there are many ways to take vitamin C, such as eating vegetables, fruits, plants, and meats of various animals, even from vitamin C tablets synthetically made. And tablets are made into several kinds, that quickly dissolving, that to be swallowed, and that to be slowly dissolving in a mouth. The tablets quickly dissolving contains calcium carbonate, sodium carbonate, etc., and not no much vitamin C consequently. Those slowly dissolving are generally made of vitamin c extracted from vegetables and fruits, containing not so high volume of vitamin C. Those to be ‘swallowed contain considerably much vitamin C, but it may’ be somewhat digested and destroyed by acid in the stomach, only a little volume may really be absorbed, hardly obtaining high effectiveness.

### SUMMARY OF THE INVENTION

An oral fast-disintegrating tablet including particles of slowly-releasable ascorbic acid contains hundreds of tiny particles dispersedly mixed with fast-disintegrating ingredient made of vitamin C mixed with quickly disintegrating substances. So it can swiftly disintegrate in a mouth, letting the tiny particles to separate from the fast-disintegrating ingredient to be mixed with saliva and then be swallowed into the stomach and then the intestines wherein the tiny particles may permit vitamin C slowly released out to be absorbed by the organs of human bodies.

Each tiny particle is composed of a nucleus of a diameter of 35-80 mesh, a layer of vitamin C mixed with a binder and a diluent agent coated around the nucleus, and then hundreds of the tiny particles are dispersedly mixed with and surrounded by the fast-disintegrating ingredient, which contains vitamin C, water excipient, microcrystalline cellulose, a diluent agent and disintegrant.

### BRIEF DESCRIPTION OF DRAWINGS AND ATTACHMENTS

This invention will be better understood by referring to the accompanying drawings and the enclosed documents

FIG. 1 is a graph of the in-vitro dissolution rates of ascorbic acid in the tablet “EVEREST” of the present invention;

FIG. 2 is a graph of the in-vitro dissolution rates of ascorbic acid in the market tablet “TAKEDA”.

FIG. 3 shows the parameters of the in-vitro dissolution rates of ascorbic acid in the tablet “EVEREST” of the present invention;

FIG. 4 shows the parameters of the in-vitro dissolution rates of ascorbic acid in the market tablet “TAKEDA”

### BRIEF DESCRIPTION OF THE PREFERRED EMBODIMENT

A preferred embodiment of an oral fast-disintegrating tablet including tiny particles of slowly-releasable ascorbic acid is composed of many tiny particles of ascorbic acid, and fast-disintegrating ingredient of ascorbic acid and other agents mixed with those tiny particles, which are dispersedly mixed with and surrounded by the fast-disintegrating ingredient.

Each tiny particle is composed of a nucleus of a diameter of 35-80 mesh, a layer of ascorbic acid containing ascorbic acid, a binder and a diluent agent, which are mixed together and then coated around the nucleus, and then a releasing control layer coated on the ascorbic acid layer.

The releasing control layer is composed of a membrane basic substance, a plastic agent, a lubricant, and the diluent agent. The membrane basic substance includes mainly at least one of hydroxypropyl cellulose, hydroxypropyl methyl cellulose, povidone, methacrylate copolymer, ethyl cellulose, hydroxypropyl methyl cellulose phthalate, and cellulose acetate phthalate, and its proportion is 40-80%.

The plastic agent uses a mixture containing chiefly one of triethyl citrate, poly-hexanediol, acetylated monoglyceride, glyceryl triacetate, castor oil, and its proportion is 10-30%.

The lubricating agent uses talc, or magnesium stearate, and its proportion is 0-40%

The diluent agent uses alcohol in the proportion of 70-95%, preferably 80%, or isopropanol or pure water in the proportion of 5-30%, preferably 20%, accounting for 5-20% of the whole weight and preferably 13%.

The fast-disintegrating ingredient is composed of ascorbic acid in the proportion of 25-45%, a water excipient in the proportion of 5-35%, microcrystalline cellulose in the proportion of 1-30%, disintegrant in the proportion of 1-10%, and a diluent agent in the proportion of 10-30%.

The water excipient uses a mixture of mainly at least one of erythritol, sortol, xylitol, mannitol, and lactose.

The disintegrant uses crosprovidone, sodium hydroxymethyl cellulose, calcium hydroxymethyl cellulose, sodium hydroxymethyl starch, or low-substituted hydroxypropyl cellulose.

The diluent agent uses a mixture of one or more of corn starch, potato starch, and pregelatinized starch.

Those components have to be sifted before use.

For manufacturing, prepare a needed amount of the membrane-coated tiny particles and the proper amount of the fast-disintegrating ingredient by weight. And the membrane-coated tiny particles preferably account for 30-70% of the whole weight, mixed with 0.1-3.0% lubricant and a 0.1-2.0% fragrance adjuster. Then a proper mold is used for shaping a tablet with the above two different components mixed, and each tablet is less than 6.0 mm thick, with a hardness of 1-5 kg so as to let the tablet able to be packaged appropriately. In case of excessive hardness, the tablet may be slow to disintegrate, and in case of soft hardness, it may break in the process of packaging.

Here is an example of practical process of manufacturing described below.

### 1. Making Tiny Particles Containing Ascorbic Acid:

Firstly, place 200 g nuclei of a diameter of 35-80 mesh in a grain spraying machine and spray on the tiny particles a mixture of ascorbic acid 535 g, hydroxypropyl cellulose 105 g, pure water 1680 g, and alcohol 1260 g preliminarily mixed together. The spraying is carried out along with drying at 60° C., and after that drying is further continued for 30 minutes at 60° C.

### 2. Coating a Membrane on the Tiny Particles:

Ethyl cellulose 294 g, hydroxypropyl methyl cellulose 21 g, ethyl citrate 21 g, talc 84 g, eatable yellow pigment No. 4 (aluminum lake) 1 g, alcohol 2520 g, and pure water 714 g are mixed and stirred evenly and then sifted through a sift of 100 mesh. Then the tiny particles with the ascorbic acid layer 800 g are placed in the grain spraying machine and sprayed with this mixed solution for coating a layer of membrane on the tiny particles. Then those tiny particles with the membrane layer is dried to contain below 2% of water, and then choose out those passing through a sift of 35-80 mesh for use.

### 3. Preparing a Fast-Integrating Ingredient:

Ascorbic acid 500 g, microcrystalline cellulose 244 g, crospovidone 54 g, mannitol 346 g, corn starch 216 g, sucralose 15 g, and silicon dioxide 4 g are mixed together after sifting by a net of 20 mesh or 40 mesh for use.

### 4. Mixing:

The tiny particles with the membrane 600 g and the fast-disintegrating ingredient 700 g are placed in an octagonal mixer for mixing them together for 10 minutes, with the lubricant and a perfume added and mixed in the above mixture for 5 minutes.

### 5. Forming Tablets:

The components finished the fourth process are made into tablets by a tablet machine, and each tablet is 16 mm in diameter, 5.4 mm±0.2 mm in thickness, and 2-5 kg in hardness.

The oral fast-disintegrating tablet acquired by the above-mentioned process has a weight 1300 mg, disintegrating in 10-30 seconds in a mouth, with the tiny particles swallowed into the stomach and intestines together with saliva and the ascorbic acid slowly releasing therein to obtain sustained releasing effect.

### Clinic Experiment

Experiments have been carried out by comparison between the drug releasing rates of each oral fast-disintegrating tablet “EVEREST” in the present invention and each market tablet “TAKEDA” under a stable condition, respectively containing 500 mg of ascorbic acid.

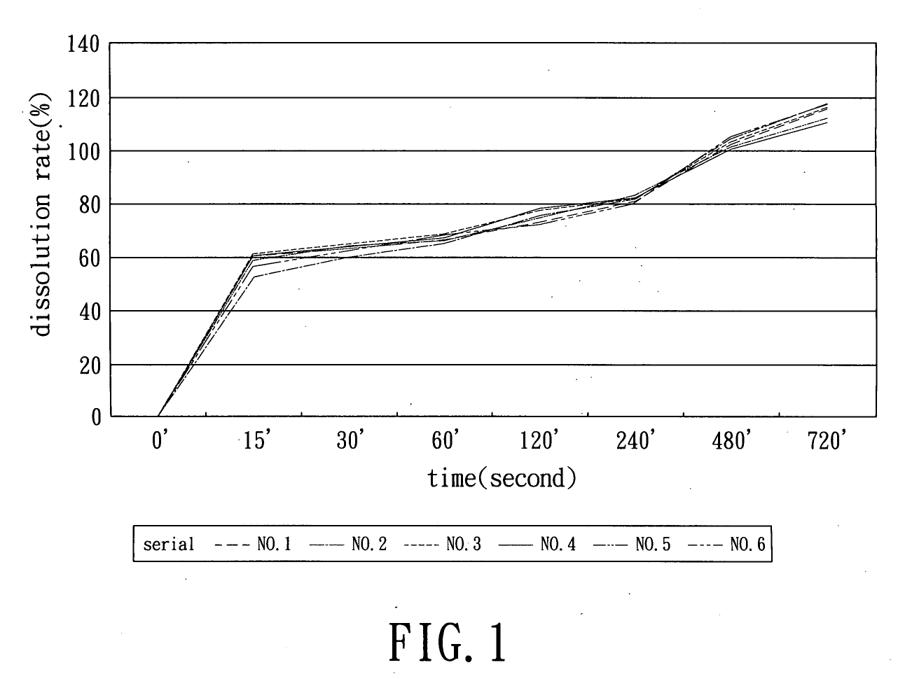

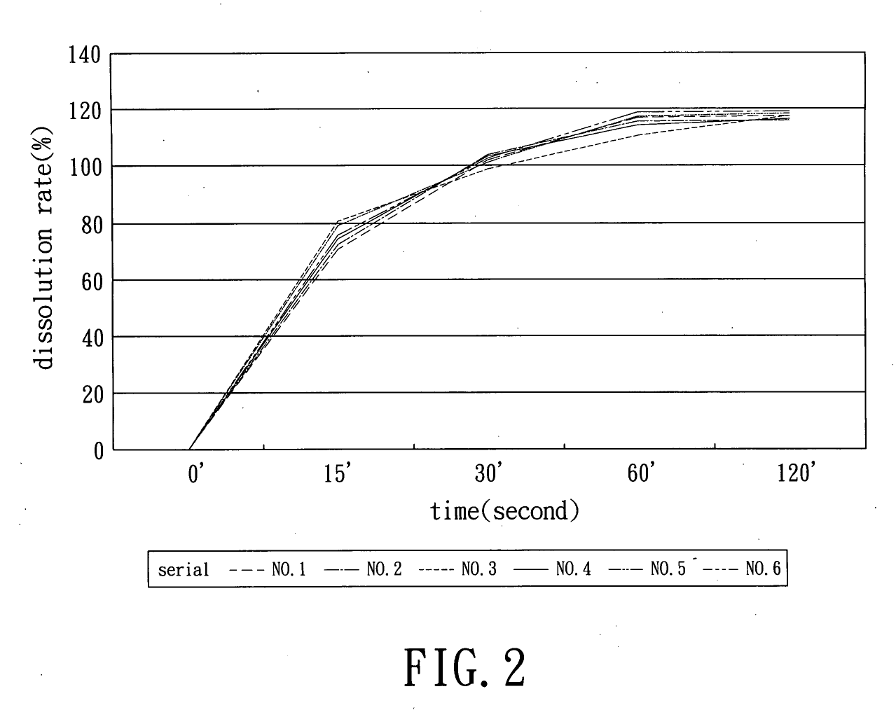

As referred to FIGS. 1 and 2, they shows the respective results of the experiments about the in-vitro dissolution rates of ascorbic acid in 6 tablets “EVEREST” in this invention and 6 market tablets “TAKEDA”.

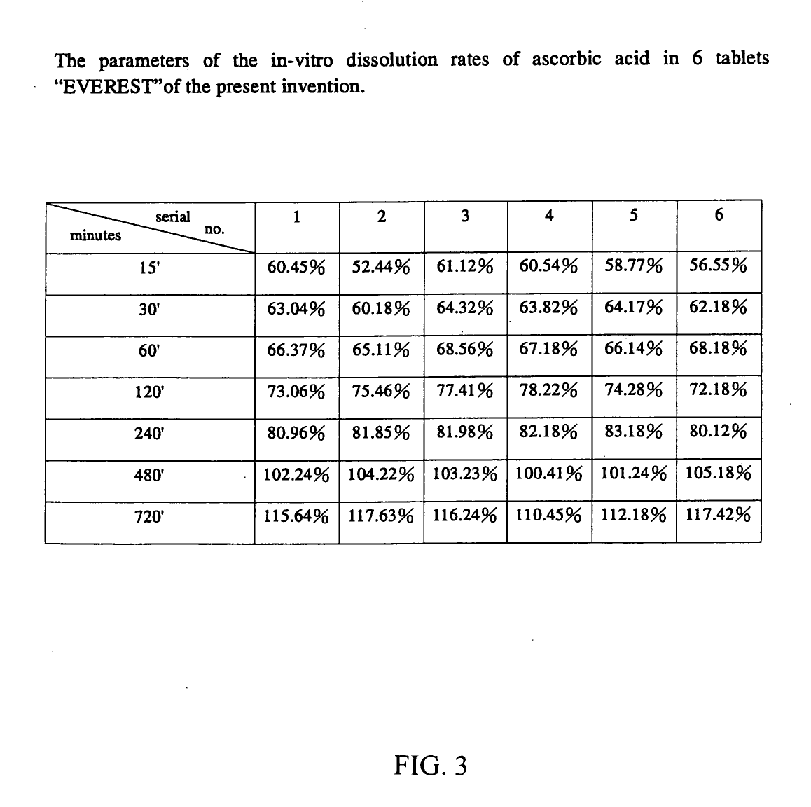

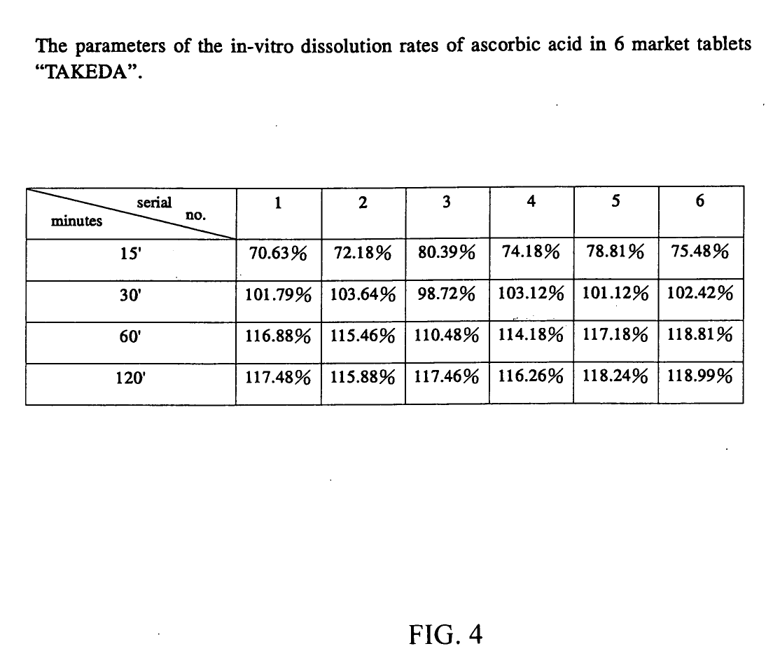

FIG. 3 shows the parameters of ascorbic acid in-vitro dissolution rates of 6 tablets “EVEREST” after 15 m (minutes), 30 m, 60 m, 120 m, 240 m, 480 m, and 720 m, and the FIG. 4 shows the parameters of ascorbic acid in-vitro dissolution rates of the market tablets “TAKEDA” after 15 m, 30 m, 60 m, and 120 m. From the FIGS. 3 and 4, it is obviously known that the oral fast-disintegrating tablets “EVEREST” of the present invention has a drug releasing function superior to those market tablets “TAKEDA”.

While the preferred embodiment of the invention has been described, it will be recognized and understood that various modifications may be made and the appended claims are intended to cover all such modifications that may fall into the spirit and scope of the invention.

What is claimed is:

1. An oral fast-disintegrating tablet including particles of slowly-releasing ascorbic acid, said tablet comprising:
Many tiny particles that are respectively composed of a nucleus and a layer of ascorbic acid coated around said nucleus, and a fast-disintegrating ingredient mixed with said tiny particles, said tiny particles dispersedly mixed and surrounded by said fast-disintegrating ingredient.

2. The oral fast-disintegrating tablet as claimed in claim 1, wherein said tiny particle comprises:
(a) said nucleus having a diameter of 35-80 mesh;
(b) a layer of ascorbic acid, said layer composed of a mixture of ascorbic acid, a binder and a diluent agent and coated on said nucleus; and,
(c) a releasing control layer composed of a membrane basic substance; a plastic agent, a lubricant and said- diluent agent mixed together, said releasing control layer slowly sprayed on said nucleus with said ascorbic acid layer.

3. The oral fast-disintegrating tablet as claimed in claim 1, wherein said fast-disintegrating ingredient contains 25-45% ascorbic acid, 5-35% water excipient, 10-30% microcrystalline cellulose, 1-10% disintegrant, and 10-30% diluent agent mixed together.

4. The oral fast-disintegrating tablet as claimed in claim 2, wherein said fast-disintegrating tablet contains 25-45% ascorbic acid, 5-35% water excipient, 10-30% microcrystalline cellulose, 1-10% disintegrant, and 10-30% diluent agent mixed together.

5. The oral fast-disintegrating tablet as claimed in claim 1, wherein a mixture of 40-50% tiny particles with a membrane and 45-60% fast-disintegrating ingredient ate mixed together and then added with a lubricant and a perfume, and said mixture with said lubricant and said perfume is made into tablets.

6. The oral fast-disintegrating tablet as claimed in claim 2, wherein a mixture of 40-50% tiny particles and 45-60% fast-disintegrating ingredient are mixed together and then added with a lubricant and a perfume, and said mixture with said lubricant and said perfume is made into tablets.

7. The oral fast-disintegrating tablet as claimed in claim 3, wherein a mixture of 40-50% tiny particles with a membrane and 45-60% fast-disintegrating ingredient are mixed and then added with a lubricant and a perfume, and said mixture with said lubricant and said perfume is made into tablets.

8. The oral fast-disintegrating tablet as claimed in claim 2, wherein said membrane basic substance of said releasing control layer contains at lease one of hydroxypropyl cellulose, hydroxypropyl methyl cellulose, povidone methacrylate copolymer, ethyl cellulose, hydroxypropyl methyl cellulose phthalate, and cellulose acetate phthalate

9. The oral fast-disintegrating tablet as claimed in claim 2, wherein said plastic agent of said releasing control layer is made of mainly at least one of triethyl citrate, poly-hexanediol, acetylated monoglyceride, glyceryl triacetate, and castor oil.

10. The oral fast-disintegrating tablet as claimed “in claim 3, wherein said water excipient of said fast-disintegrating ingredient is made of mainly least one of erythritol, sortol, xylitol, mannitol, and lactose.

11. The oral fast-disintegrating tablet as claimed in claim 4, wherein said water excipient of said fast-disintegrating ingredient is made of mainly at least one of erythritol, sortol, xylitol, mannitol, and lactose.

12. The oral fast-disintegrating tablet as claimed in claim 5, said tablet has hardness in the scope of 1-5 kg.

13. The oral fast-disintegrating tablet as claimed in claim 6, wherein said tablet has hardness in the scope of 1-5 kg.

14. The oral fast-disintegrating tablet as claimed in claim 7, wherein said tablet has hardness in the scope of 1-5 kg.
